(19) United States
(12) Patent Application Publication
(10) Pub. No.: US 2018/0327865 A1
(43) Pub. Date: Nov. 15, 2018

(54) METHOD FOR NEXT GENERATION SEQUENCING BASED GENETIC TESTING

(71) Applicant: City University of Hong Kong, Kowloon (HK)

(72) Inventors: Zicheng Zhao, Kowloon Tong (HK); Bowen Tan, Tai Wai (HK); Shuai Cheng Li, Ma On San (HK)

(21) Appl. No.: 15/590,120

(22) Filed: May 9, 2017

(51) Int. Cl. C12Q 1/68; G06F 19/14; G06F 19/24

(52) U.S. Cl. CPC C12Q 1/6888; C12Q 2600/156; G06F 19/24; G06F 19/14

(57) ABSTRACT

A next generation sequencing (NGS) based method includes applying, for one or more genetic loci, respective NGS data for genotype of a first subject, genotype of a second subject, and genotype of an alleged offspring of the first and second subjects to a statistical model calculating a value representing a likelihood the offspring is a true offspring of the first and second subjects. The NGS data includes genotype and sequencing read of the first tested subject; genotype and sequencing read of the second tested subject; and genotype and sequencing read of the alleged offspring. The statistical model utilizes a probability of the genotype of the first tested subject in a subject population; a probability of the genotype of the second tested subject in a subject population; and a probability of the genotype of the alleged offspring in a subject population.

### TECHNICAL FIELD

The present invention relates to a next generation sequencing (NGS) based method for genetic testing and particularly, although not exclusively, to a next generation sequencing (NGS) based method for paternity testing.

### BACKGROUND

Paternity testing has experienced great changes in the last three decades as a result of improvement of DNA sequencing technologies. To date, the most widely adopted methods for paternity testing in forensic laboratories worldwide are polymerase chain reaction (PCR) based sequencing and capillary electrophoresis (CE) based sequencing for detection of fragment length variations in 13 core short tandem repeat (STR) markers in the Combined DNA Index System (CODIS) published by the Federal Bureau of Investigation (FBI).

Although the CODIS system is powerful and widely applied, it suffers a number of problems. As an increasing number of forensic databases based on CODIS core STRs are established worldwide, the sizes of the databases are dramatically enlarged, and so there probability of random hits (“cold hits”) in databases would also dramatically increase. In applications such as individual identification in criminal cases, this may cause an individual in the forensic database to be falsely charged as the criminal when a new crime occurs. On the other hand, in applications such as paternity testing, the result is vulnerable to false exclusions caused by allelic dropout, null alleles, contamination, human errors and mutations in offspring.

### SUMMARY OF THE INVENTION

In accordance with a first aspect of the present invention, there is provided a next generation sequencing (NGS) based method for genetic testing, comprising: applying, for one or more genetic loci, respective NGS data related to genotype of a first tested subject, genotype of a second tested subject, and genotype of an alleged offspring of the first and second tested subjects to a statistical model for calculating a value representing a likelihood that the alleged offspring is a true offspring of the first and second subjects; and determining, based on the respective values calculated for the one or more genetic loci, a likelihood that the alleged offspring is a true offspring of the first and second tested subjects; wherein the NGS data includes: genotype and sequencing read of the first tested subject; genotype and sequencing read of the second tested subject; and genotype and sequencing read of the alleged offspring; wherein the statistical model utilizes: a probability of the genotype of the first tested subject in a subject population; a probability of the genotype of the second tested subject in a subject population; and a probability of the genotype of the alleged offspring in a subject population.

In one embodiment of the first aspect, the method is applied to a plurality of genetic loci.

In one embodiment of the first aspect, the statistical model utilizes the respective probability of the genotype of the first tested subject, the second tested subject, and the alleged offspring as posterior probability with the sequencing read of the first tested subject, the second tested subject, and the alleged offspring.

In one embodiment of the first aspect, the statistical model applies the following for calculating the value of the respective genetic loci:

$$\frac{{\sum }_{{g}_{c} ; {g}_{m} , {g}_{af}} T ( {g}_{c} \vert {g}_{m} , {g}_{af} ) P ( {g}_{c} \vert {D}_{c} ) P ( {g}_{m} \vert {D}_{m} ) P ( {g}_{af} \vert {D}_{af} )}{{\sum }_{{g}_{c} , {g}_{m}} T ( {g}_{c} \vert {g}_{m} ) P ( {g}_{c} \vert {D}_{c} ) P ( {g}_{m} \vert {D}_{m} )}$$

where Dₘ represents the sequencing read for the first tested subject, D_{c} represents the sequencing read for the alleged offspring, D_{af} represents the sequencing read for the second tested subject, gₘ represents the genotype for a corresponding locus of the first tested subject, g_{c} represents the genotype for a corresponding locus of the alleged offspring, g_{af} represents the genotype for a corresponding locus of the second tested subject, T(g_{c}|gₘ, g_{af}) represents a likelihood that both alleles of the alleged offspring are inherited from the first and second tested subjects, and T(g_{c}|g_{mf}) represents a likelihood that the tested second subject is not biologically related to the alleged offspring.

In one embodiment of the first aspect, the first tested subject is a mother of the offspring and the second tested subject is an alleged father of the offspring.

In one embodiment of the first aspect, the method further comprises the step of obtaining raw NGS data from the first tested subject, the second tested subject, and the alleged offspring.

In one embodiment of the first aspect, in the raw NGS data, a sequencing coverage of the first tested subject is above or equal to 0.5×. In other words, the method of the first aspect would operate reliably even if the raw NGS data of the first tested subject is sub-sampled to a certain extent.

In one embodiment of the first aspect, in the raw NGS data a sequencing coverage of the second tested subject is above or equal to 0.5×. In other words, the method of the first aspect would operate reliably even if the raw NGS data of the second tested subject is sub-sampled to a certain extent.

In one embodiment of the first aspect, in the raw NGS data a sequencing coverage of the alleged offspring is above or equal to 0.5×. In other words, the method of the first aspect would operate reliably even if the raw NGS data of the alleged offspring is sub-sampled to a certain extent.

In one embodiment of the first aspect, the method further comprises: prior to the application step, filtering raw NGS data to remove marker with more than two alleles to obtain the respective NGS data for the one or more genetic loci.

In one embodiment of the first aspect, the method further comprises the steps of: dividing respective genomes in the corresponding NGS data of the first tested subject, the second tested subject, and the alleged offspring into a plurality of segments; sorting markers in each of the plurality of segments based on a probability of exclusion; and selecting a plurality of markers based on the sorting result for application to the statistical model. Preferably, the selection step comprises selecting a plurality of markers with the highest probability of exclusion.

In accordance with a second aspect of the present invention, there is provided a next generation sequencing (NGS) based system for genetic testing, comprising: means for applying, for one or more genetic loci, respective NGS data related to genotype of a first tested subject, genotype of a second tested subject, and genotype of an alleged offspring of the first and second tested subjects to a statistical model for calculating a value representing a likelihood that the alleged offspring is a true offspring of the first and second subjects; and means for determining, based on the respective values calculated for the one or more genetic loci, a likelihood that the alleged offspring is a true offspring of the first and second tested subjects; wherein the NGS data includes: genotype and sequencing read of the first tested subject; genotype and sequencing read of the second tested subject; and genotype and sequencing read of the alleged offspring; wherein the statistical model utilizes: a probability of the genotype of the first tested subject in a subject population; a probability of the genotype of the second tested subject in a subject population; and a probability of the genotype of the alleged offspring in a subject population.

In one embodiment of the second aspect, the statistical model utilizes the respective probability of the genotype of the first tested subject, the second tested subject, and the alleged offspring as posterior probability with the sequencing read of the first tested subject, the second tested subject, and the alleged offspring.

In one embodiment of the second aspect, the statistical model applies the following for calculating the value of the respective genetic loci:

$$\frac{{\sum }_{{g}_{c} ; {g}_{m} , {g}_{af}} T ( {g}_{c} \vert {g}_{m} , {g}_{af} ) P ( {g}_{c} \vert {D}_{c} ) P ( {g}_{m} \vert {D}_{m} ) P ( {g}_{af} \vert {D}_{af} )}{{\sum }_{{g}_{c} , {g}_{m}} T ( {g}_{c} \vert {g}_{m} ) P ( {g}_{c} \vert {D}_{c} ) P ( {g}_{m} \vert {D}_{m} )}$$

where Dₘ represents the sequencing read for the first tested subject, D_{c} represents the sequencing read for the alleged offspring, D_{af} represents the sequencing read for the second tested subject, gₘ represents the genotype for a corresponding locus of the first tested subject, g_{c} represents the genotype for a corresponding locus of the alleged offspring, g_{af} represents the genotype for a corresponding locus of the second tested subject, T(g_{c}|gₘ, g_{af}) represents a likelihood that both alleles of the alleged offspring are inherited from the first and second tested subjects, and T(g_{c}|g_{mf}) represents a likelihood that the tested second subject is not biologically related to the alleged offspring.

In one embodiment of the second aspect, the first tested subject is a mother of the offspring and the second tested subject is an alleged father of the offspring.

In some embodiments of the second aspect, the system may also include structures suitable for implementing the method in various embodiments of the first aspect.

In accordance with a third aspect of the present invention, there is provided a non-transitory computer readable medium for storing computer instructions that, when executed by one or more processors, causes the one or more processors to perform a next generation sequencing (NGS) based method for genetic testing, comprising: applying, for one or more genetic loci, respective NGS data related to genotype of a first tested subject, genotype of a second tested subject, and genotype of an alleged offspring of the first and second tested subjects to a statistical model for calculating a value representing a likelihood that the alleged offspring is a true offspring of the first and second subjects; and determining, based on the respective values calculated for the one or more genetic loci, a likelihood that the alleged offspring is a true offspring of the first and second tested subjects; wherein the NGS data includes: genotype and sequencing read of the first tested subject; genotype and sequencing read of the second tested subject; and genotype and sequencing read of the alleged offspring; wherein the statistical model utilizes: a probability of the genotype of the first tested subject in a subject population; a probability of the genotype of the second tested subject in a subject population; and a probability of the genotype of the alleged offspring in a subject population.

In some embodiments of the third aspect, the non-transitory computer readable medium may contain computer instructions that, when executed by one or more processors, causes the one or more processors to perform the method in some embodiments of the first aspect.

### BRIEF DESCRIPTION OF THE DRAWINGS

Embodiments of the present invention will now be described, by way of example, with reference to the accompanying drawings in which:

FIG. 1 is a next generation sequencing based method for genetic testing in accordance with embodiment of the present invention;

FIG. 2 is an information handling system suitable for implementing the method of FIG. 1 in accordance with one embodiment of the present invention;

FIG. 3 is a graph showing experimental results of the paternity index for true trio and false trio in each partition window obtained using the method of the present invention;

FIG. 4A is a graph showing experimental results for differentiating between true trio and false trio using the method of the present invention with a sequencing coverage of ˜2×;

FIG. 4B is a graph showing experimental results for differentiating between true trio and false trio using the method of the present invention with a sequencing coverage of ˜1×;

FIG. 4C is a graph showing experimental results for differentiating between true trio and false trio using the method of the present invention with a sequencing coverage of ˜0.5×; and

FIG. 4D is a graph showing experimental results for differentiating between true trio and false trio using the method of the present invention with a sequencing coverage of ˜0.3×.

### DETAILED DESCRIPTION OF THE PREFERRED EMBODIMENT

The inventors of the present invention have devised, through research, experiments, and trials, that next-generation sequencing (NGS), with its high-throughput and relatively low cost compared to other sequencing techniques, may provide enormous potential feasibilities in forensic studies. From the first pyro-sequencing-based high-throughput sequencing system—the 454 Genome Sequencing System, introduced by Roche in 2005, the NGS technique gradually matures through time. The throughput of a single sequencing run nowadays has increased significantly and the cost-per-base has reduced significantly. For paternity testing, whole genome sequencing provides redundant marker information that is capable of handling complex scenarios with high accuracy.

The inventors of the present invention have also devised, through research, experiments, and trials, that in order to acquire a reliable result with low cost, a minimum requirement of sequencing coverage must be set using NGS-based methods and systems. However, when the sequencing coverage is low, genotypes of the tested individuals are associated with statistical uncertainty, for mainly two reasons. First, for haploids, both alleles may not be samples. Second, in most NGS data, the error rate is at least 0.1% even after filtering out base pairs with low quality. This may result in many homozygous loci wrongly inferred as heterozygous.

The most widely applied method for paternity testing nowadays is the likelihood method. Given the genotypes of a tested trio, this method relies on the calculation of the likelihood ratio of two hypotheses called Paternity Index (PI):

(1) X, the likelihood of the tested man is the biological father of the child (True Trio);
(2) Y, the likelihood of a random man is the biological father of the child (False Trio); For each locus, denote g_{q}f, gₘ and g_{c}, as the genotypes for the alleged father, mother and child respectively, then the PI value can be written as

$$PI = \frac{T ( {g}_{c} \vert {g}_{m} , {g}_{a} )}{T ( {g}_{c} \vert {g}_{m} )} \qquad ( 1 )$$

where T(g_{c}|gₘ, g_{af}) is the likelihood of true trio, which means that both alleles of the child are inherited from the mother and the alleged father; T(g_{c}|g_{mf}) is the likelihood of that the tested man is not the biological father of the child.

Ranges from 0 to infinity, the PI value provides DNA evidence of paternity for each locus. Specifically, if PI>1, then it indicates that the genetic evidence of this locus supports that the tested man is the biological father; if PI=1, then it indicates that the genetic evidence of this locus provides no information on paternity; and if PI<1, then it indicates that the genetic evidence of this locus is more consistent with non-paternity than paternity. Low PI values are primarily resulting from inconsistency in genetic markers, which may be caused by non-paternity, mutations in offspring and wrong genotype calls by sequencing errors.

The following embodiment of the present invention provides a method that reduces the errors caused by sequencing errors.

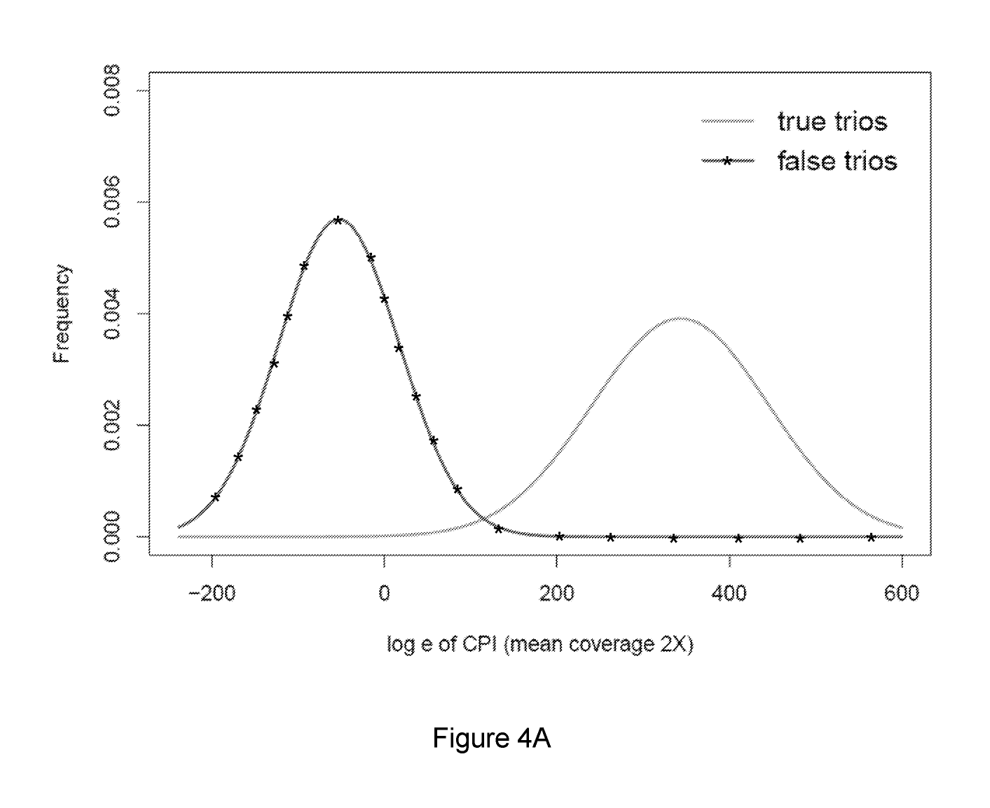

FIG. 1 shows a block diagram illustrating the genetic testing method 100 based on NGS in one embodiment of the present invention. The method begins in step 102, which involves applying, for each genetic loci, respective NGS data related to genotype of a first tested subject, genotype of a second tested subject, and genotype of an alleged offspring of the first and second tested subjects to a statistical model. Then, in step 104, the method 100 calculates, for each genetic loci, a value representing a likelihood that the alleged offspring is a true offspring of the first and second subjects. In step 106, the method determines, based on the respective values calculated, a likelihood that the alleged offspring is a true offspring of the first and second tested subjects. In the present embodiment, the NGS data includes genotype and sequencing read of, respectively, the first tested subject, the second tested subject, and the alleged offspring. The statistical model uses, in addition to the NGS data, a probability of the genotype of the first tested subject in a subject population, a probability of the genotype of the second tested subject in a subject population, and a probability of the genotype of the alleged offspring in a subject population. Genetic data associated with the subject population may be taken from a genetic database containing genomes of individuals. The method 100 is preferably applied to multiple genetic loci, but in some cases, it may be applied to only one genetic locus.

Preferably, in the method 100 of the present embodiments, the statistical model utilizes the respective probability of the genotype of the first tested subject, the second tested subject, and the alleged offspring as posterior probability with the sequencing read of the first tested subject, the second tested subject, and the alleged offspring.

In some examples, the method 100 may further include obtaining raw NGS data from the first tested subject, the second tested subject, and the alleged offspring. This is prior to step 102. Preferably, in the raw NGS data, respective sequencing coverage of the first tested subject, the second tested subject, and the alleged offspring are each as low as 0.5×. In one example, in the raw NGS data, respective sequencing coverage of the first tested subject, the second tested subject, and the alleged offspring are each between 0.5× and 2×. The method may further include, prior to step 102, filtering, either automatically or manually, raw NGS data to remove marker with more than two alleles to obtain the respective NGS data for the genetic loci.

In one embodiment, the method 100 may include dividing respective genomes in the corresponding NGS data of the first tested subject, the second tested subject, and the alleged offspring into a plurality of segments. The markers in each of the plurality of segments are then sorted based on a probability of exclusion, and afterwards, one or more markers may be selected based on the sorting result for application to the statistical model. In one example, the markers with the highest probability of exclusion are selected.

In the method 100 of the present embodiment, to reduce the errors caused by sequencing errors, the probability of the genotypes are modelled as the posterior probability with the observed reads by Bayesian rule. In the present embodiment, the PI value is defined as

$$\frac{{\sum }_{{g}_{c} ; {g}_{m} , {g}_{af}} T ( {g}_{c} \vert {g}_{m} , {g}_{af} ) P ( {g}_{c} \vert {D}_{c} ) P ( {g}_{m} \vert {D}_{m} ) P ( {g}_{af} \vert {D}_{af} )}{{\sum }_{{g}_{c} , {g}_{m}} T ( {g}_{c} \vert {g}_{m} ) P ( {g}_{c} \vert {D}_{c} ) P ( {g}_{m} \vert {D}_{m} )} \qquad ( 2 )$$

where D_{c}, Dₘ and D_{af} represent the observed sequencing reads for, respectively, the tested offspring, mother and alleged father.

According to the Bayesian rule, the conditional probability of the individuals real genotype is g_{i,j} with allele i and j given the observed read on such locus is

$$P ( {g}_{i , j} \vert D ) = \frac{P ( {g}_{i , j} ) P ( D \vert {g}_{i , j} )}{P ( D )} = \frac{P ( {g}_{i , j} ) P ( D \vert {g}_{i , j} )}{{\sum }_{i , j} P ( {g}_{i , j} ) P ( D \vert {g}_{i , j} )} \qquad ( 3 )$$

where P(g_{i,j}) is the genotype frequency in the subject population. Under the assumption of Hardy-Weinberg equilibrium, it can be calculated that

$$P ( {g}_{i , j} ) = \{ \begin{matrix}f ( i ) f ( j ) & if i = j \\ 2 f ( i ) f ( j ) & if i \neq j\end{matrix} \qquad ( 4 )$$

where f(i) and f(j) are the allele frequencies for allele i and j respectively.

In the method of the present embodiment, P(D|g_{i,j}) is the likelihood of observing the allele type that are supported by reads if the genotype is g_{i,j}. Assuming that the reads are independent of each other in the sequencing process, then

P(D|g_{i,j})=ΠₖP(dₖ|g_{i,j}) (5

where dₖ is the k-th read that covers the corresponding locus.

The present embodiment models the sequencing process as a random process following binomial distribution, which means the probabilities of a sequenced read from both alleles are equal. Thus

P(D|g_{i,j})=C_{D}^{d}^{ᵢ}p(l|g_{i,j})^{d}^{ᵢ}p(j|g_{i,j})^{d}^{ⱼ} (6)

where P(i|g_{i,j}) is the probability of the sequenced read with allele i in one sampling under the condition that the individual genotype is g_{i,j}. In one example, if g_{i,j} is Aa, then p(A|g_{Aa})=p(a|g_{Aa})=0.5.

Considering sequencing errors, and denote the observed reads for allele i and j as dᵢ and dⱼ respectively, the real reads (without error) of allele i and j as rᵢ and rᵢ. Then it can be determined that

P(dᵢ,dⱼ|g_{i,j})=Σᵣ_{ᵢ}_{,r}_{ⱼ}_{,e}P(rᵢ,rⱼ,e|g_{i,j}) (7)

Suppose in one example it is observed that there are 4 reads supporting allele i and 6 reads supporting allele j for an SNP locus, the real situation may be 4 reads for i and 6 reads for j without sequencing error, or 3 reads for i and 7 reads for j with 1 sequencing error. If the real situation is 4 reads for i and 6 reads for j, the number of errors may be 0, 2, 4, . . . In other words, in this example, there must be even opposite errors, i.e., if one read is incorrectly sequenced as i instead of j, there must be another error where allele j is sequenced as allele i in order to get the final observation.

To convert the theoretically sequencing scenario (without sequencing errors) to the observed case (with sequencing errors), the minimum number of sequencing errors on this locus is eₘᵢₙ=|dᵢ−rᵢ|=|dⱼ−rⱼ|. Under the assumption that each read can only be incorrectly sequenced once, the total error number on this locus e must satisfy

$$\{ \begin{matrix}\vert {r}_{i} - e \vert \leq {d}_{i} \\ \vert {r}_{j} - e \vert \leq {d}_{i}\end{matrix} \qquad ( 8 )$$

After clarifying the rules for errors, equation (7) may be expanded by listing out all the cases with sequencing errors. Subsequently,

P(D|g_{i,j})=Σ_{d}_{ᵢ}_{,d}_{ⱼ}_{,e}P(dᵢ,dⱼ,e|g_{i,j}) (9)

where e is subject to inequality set in equations (8).

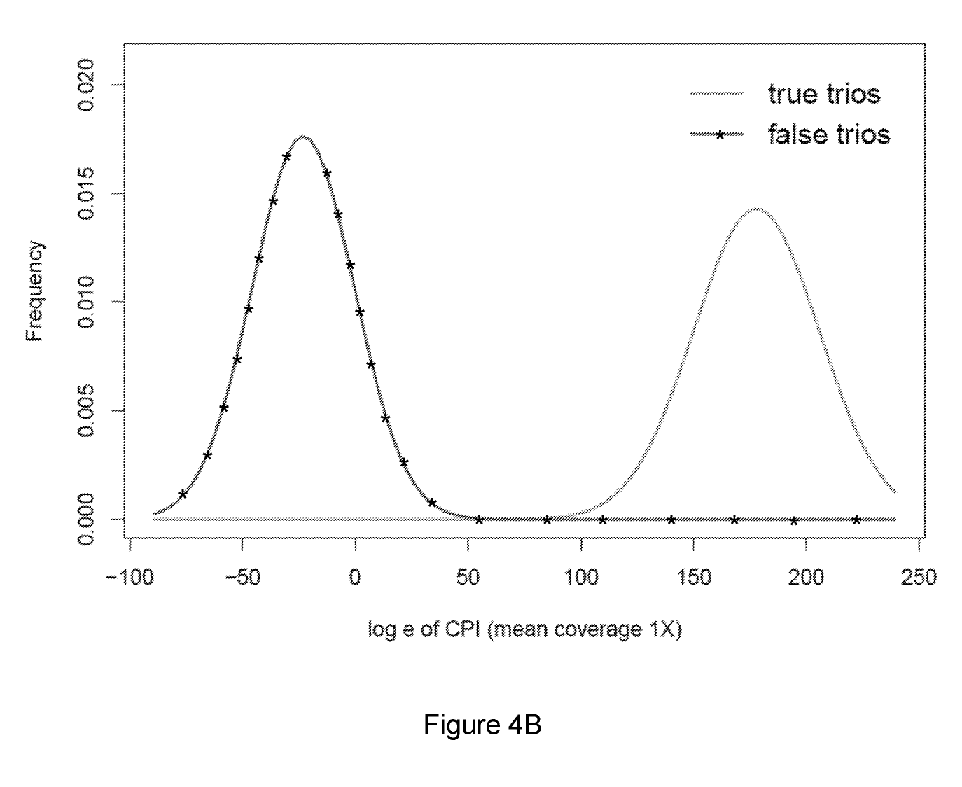

Referring to FIG. 2, there is shown a schematic diagram of an exemplary information handling system 200 that can be used as for implementing the method 100 of FIG. 1 in one embodiment of the present invention. Preferably, the information handling system 200 may have different configurations, and it generally comprises suitable components necessary to receive, store and execute appropriate computer instructions or codes. The main components of the information handling system 200 are a processing unit 202 and a memory unit 204. The processing unit 202 is a processor such as a CPU, an MCU, etc. The memory unit 204 may include a volatile memory unit (such as RAM), a non-volatile unit (such as ROM, EPROM, EEPROM and flash memory) or both. The memory 204 may be sorted with the NGS data, the raw NGS data, and/or the processed result. Preferably, the information handling system 200 further includes one or more input devices 206 such as a keyboard, a mouse, a stylus, a microphone, a tactile input device (e.g., touch sensitive screen) and a video input device (e.g., camera). The information handling system 200 may further include one or more output devices 208 such as one or more displays, speakers, disk drives, and printers. The displays may be a liquid crystal display, a light emitting display or any other suitable display that may or may not be touch sensitive. The information handling system 200 may further include one or more disk drives 212 which may encompass solid state drives, hard disk drives, optical drives and/or magnetic tape drives. A suitable operating system may be installed in the information handling system 200, e.g., on the disk drive 212 or in the memory unit 204 of the information handling system 200. The memory unit 204 and the disk drive 212 may be operated by the processing unit 202. The information handling system 200 also preferably includes a communication module 210 for establishing one or more communication links (not shown) with one or more other computing devices such as a server, personal computers, terminals, wireless or handheld computing devices. The communication module 210 may be a modem, a Network Interface Card (NIC), an integrated network interface, a radio frequency transceiver, an optical port, an infrared port, a USB connection, or other interfaces. The communication links may be wired or wireless for communicating commands, instructions, information and/or data. In one example the information handling system 200 can download or retrieve genetic data related to the subject population through the communication module 210. Alternatively or additionally, the information handling system 200 can transfer processed result to a network, a server, etc., through the communication module 210. Preferably, the processing unit 202, the memory unit 204, and optionally the input devices 206, the output devices 208, the communication module 210 and the disk drives 212 are connected with each other through a bus, a Peripheral Component Interconnect (PCI) such as PCI Express, a Universal Serial Bus (USB), and/or an optical bus structure. In one embodiment, some of these components may be connected through a network such as the Internet or a cloud computing network. A person skilled in the art would appreciate that the information handling system 200 shown in FIG. 2 is merely exemplary, and that different information handling system 200 may have different configurations and still be applicable in the present invention.

To verify the performance of the method in the above embodiments of the present invention, the following experiments are performed.

One experiment uses genetic data of 320 Chinese individuals in 1000 Genome Project Phase 3. In the experiment, the allele frequencies for both SNP and STR markers in Chinese sub-population were counted. Then, 8 Chinese family trio NGS data with average sequencing coverage of ˜32× were collected. After stringently filtering out the markers with more than two alleles, the statistical model in the above embodiments of the present invention is applied.

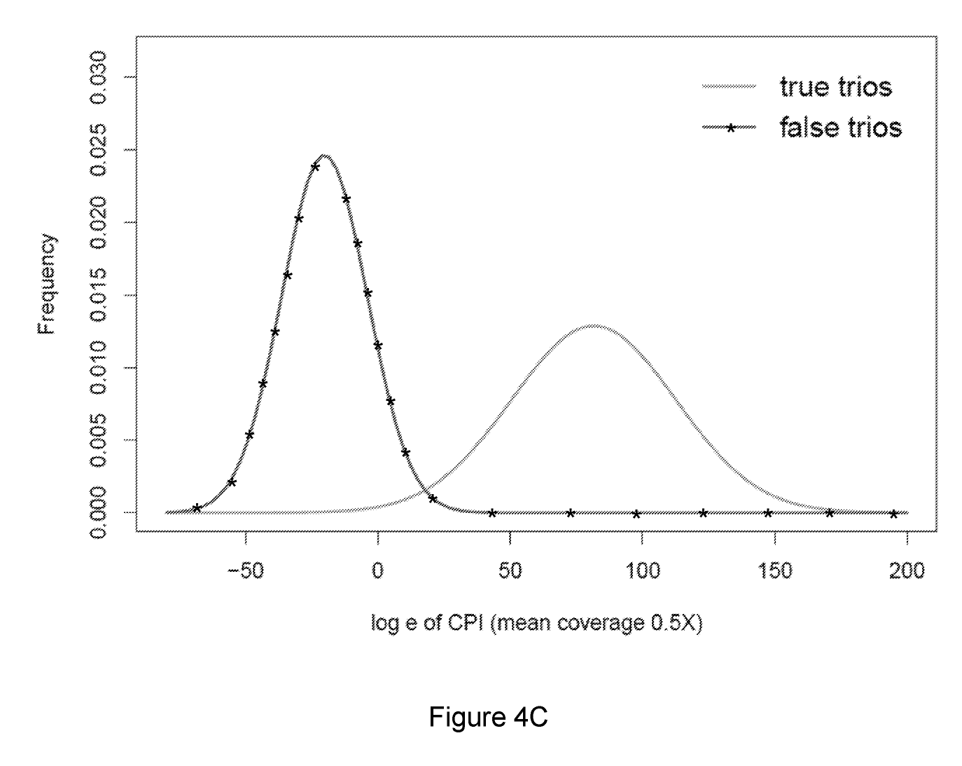

FIG. 3 shows an example for a true trio and a false trio determined using the proposed method. In the method, the human genome is first divided by 10-Mb windows without overlaps. In each window, all of the markers were sorted by the probability of exclusion (PE) and the first ten markers were selected as the genetic evidence of paternity for the window. In FIG. 3, the log PI values for the true trio in all partitions are larger than 0 (above the chromosome line), supporting the conclusion that the tested man is the biological father of the child. Contrarily, most of the log PI values for the false trio are negative (below chromosome line). The logs of combined paternity index (CPI) for the true trio and the false trio are 1,841.28 and −2,418.12 respectively. It can be seen that the method of the present embodiments can justify paternity with high confidence.

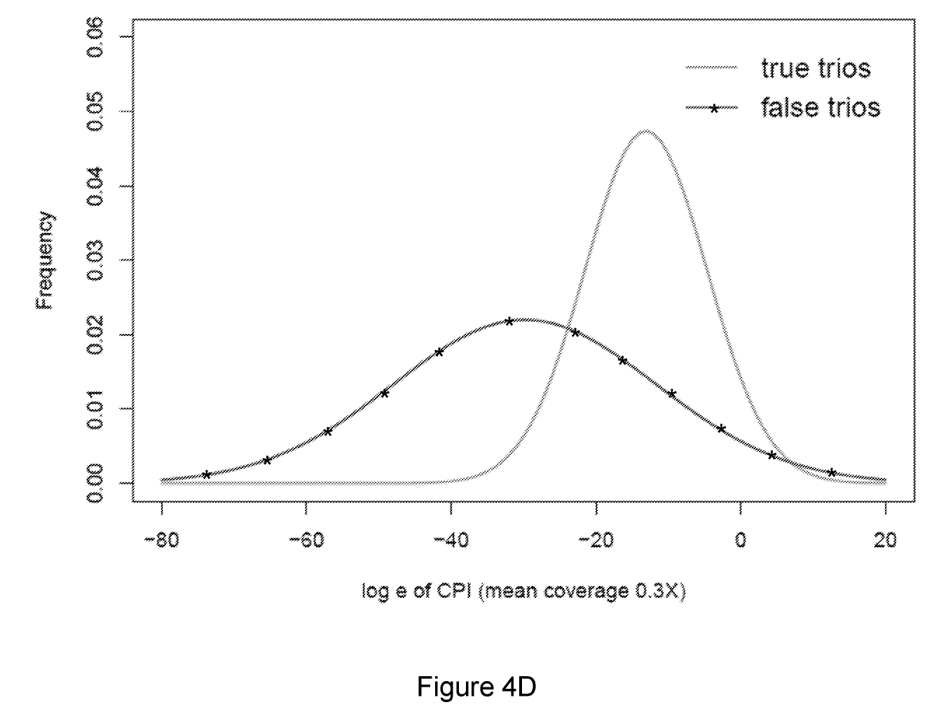

A further experiment was performed by randomizing subsample reads to reduce the sequencing coverage of samples. In this experiment, the overall coverage was reduced to ˜2×, ˜1×, ˜0.5× and ˜0.3× respectively. With each sequencing coverage, 800 experiments for both true trio and false trio (each family trio 100 times) were processed. As shown in FIGS. 4A-4D, when the sequencing coverage decreased to about 0.5×, the method of the present embodiments can still effectively separate true trio with the false trio with high accuracy.

Embodiments of the present invention have provided a statistical model based method for genetic testing with NGS data. By considering the probability of sequencing errors and missing alleles, the likelihood of the genotypes for individuals in the tested trio is calculated, and is then combined together to obtain the overall probability that the tested subject is biologically related to the alleged offspring (e.g., the tested man is the true biological father of the alleged offspring). The method in some embodiments of the present invention requires the minimum 0.5×NGS sequencing data of a trio family to perform accurate determination. As a result, reliable result can be obtained with relatively low cost.

It should be noted that the methods of the present invention can be applied not only to paternity testing, but also to genetic analysis for individual identification. Also, the present invention is not limited in its application to human beings, but may also apply to other animal, plants, etc.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A next generation sequencing (NGS) based method for genetic testing, comprising:
applying, for one or more genetic loci, respective NGS data related to genotype of a first tested subject, genotype of a second tested subject, and genotype of an alleged offspring of the first and second tested subjects to a statistical model for calculating a value representing a likelihood that the alleged offspring is a true offspring of the first and second subjects; and
determining, based on the respective values calculated for the one or more genetic loci, a likelihood that the alleged offspring is a true offspring of the first and second tested subjects;
wherein the NGS data includes:
genotype and sequencing read of the first tested subject;
genotype and sequencing read of the second tested subject; and
genotype and sequencing read of the alleged offspring;
wherein the statistical model utilizes:
a probability of the genotype of the first tested subject in a subject population;
a probability of the genotype of the second tested subject in a subject population; and
a probability of the genotype of the alleged offspring in a subject population.

2. The method of claim 1, wherein the method is applied to a plurality of genetic loci.

3. The method of claim 1, wherein the statistical model utilizes the respective probability of the genotype of the first tested subject, the second tested subject, and the alleged offspring as posterior probability with the sequencing read of the first tested subject, the second tested subject, and the alleged offspring.

4. The method of claim 1, wherein the statistical model applies the following for calculating the value of the respective genetic loci:
$$\frac{{\sum }_{{g}_{c} ; {g}_{m} , {g}_{af}} T ( {g}_{c} \vert {g}_{m} , {g}_{af} ) P ( {g}_{c} \vert {D}_{c} ) P ( {g}_{m} \vert {D}_{m} ) P ( {g}_{af} \vert {D}_{af} )}{{\sum }_{{g}_{c} , {g}_{m}} T ( {g}_{c} \vert {g}_{m} ) P ( {g}_{c} \vert {D}_{c} ) P ( {g}_{m} \vert {D}_{m} )}$$
where
Dₘ represents the sequencing read for the first tested subject,
D_{c} represents the sequencing read for the alleged offspring,
D_{af} represents the sequencing read for the second tested subject,
gₘ represents the genotype for a corresponding locus of the first tested subject,
g_{c} represents the genotype for a corresponding locus of the alleged offspring,
g_{af} represents the genotype for a corresponding locus of the second tested subject,
T(g_{c}|gₘ, g_{af}) represents a likelihood that both alleles of the alleged offspring are inherited from the first and second tested subjects, and
T(g_{c}|g_{mf}) represents a likelihood that the tested second subject is not biologically related to the alleged offspring.

5. The method of claim 4, wherein first tested subject is a mother of the offspring and the second tested subject is an alleged father of the offspring.

6. The method of claim 1, further comprising the step of:
obtaining raw NGS data from the first tested subject, the second tested subject, and the alleged offspring.

7. The method of claim 6, wherein in the raw NGS data, a sequencing coverage of the first tested subject is above or equal to 0.5×.

8. The method of claim 6, wherein in the raw NGS data a sequencing coverage of the second tested subject is above or equal to 0.5×.

9. The method of claim 6, wherein in the raw NGS data a sequencing coverage of the alleged offspring is above or equal to 0.5×.

10. The method of claim 6, further comprising:
prior to the application step, filtering raw NGS data to remove marker with more than two alleles to obtain the respective NGS data for the one or more genetic loci.

11. The method of claim 1, further comprising:
dividing respective genomes in the corresponding NGS data of the first tested subject, the second tested subject, and the alleged offspring into a plurality of segments;
sorting markers in each of the plurality of segments based on a probability of exclusion;
selecting a plurality of markers based on the sorting result for application to the statistical model.

12. The method of claim 11, wherein the selection step comprises:
selecting a plurality of markers with the highest probability of exclusion.

13. A next generation sequencing (NGS) based system for genetic testing, comprising:
means for applying, for one or more genetic loci, respective NGS data related to genotype of a first tested subject, genotype of a second tested subject, and genotype of an alleged offspring of the first and second tested subjects to a statistical model for calculating a value representing a likelihood that the alleged offspring is a true offspring of the first and second subjects; and
means for determining, based on the respective values calculated for the one or more genetic loci, a likelihood that the alleged offspring is a true offspring of the first and second tested subjects;
wherein the NGS data includes:
genotype and sequencing read of the first tested subject;
genotype and sequencing read of the second tested subject; and
genotype and sequencing read of the alleged offspring;
wherein the statistical model utilizes:
a probability of the genotype of the first tested subject in a subject population;
a probability of the genotype of the second tested subject in a subject population; and
a probability of the genotype of the alleged offspring in a subject population.

14. The system of claim 13, wherein the statistical model utilizes the respective probability of the genotype of the first tested subject, the second tested subject, and the alleged offspring as posterior probability with the sequencing read of the first tested subject, the second tested subject, and the alleged offspring.

15. The system of claim 13, wherein the statistical model applies the following for calculating the value of the respective genetic loci:
$$\frac{{\sum }_{{g}_{c} ; {g}_{m} , {g}_{af}} T ( {g}_{c} \vert {g}_{m} , {g}_{af} ) P ( {g}_{c} \vert {D}_{c} ) P ( {g}_{m} \vert {D}_{m} ) P ( {g}_{af} \vert {D}_{af} )}{{\sum }_{{g}_{c} , {g}_{m}} T ( {g}_{c} \vert {g}_{m} ) P ( {g}_{c} \vert {D}_{c} ) P ( {g}_{m} \vert {D}_{m} )}$$
where
Dₘ represents the sequencing read for the first tested subject,
D_{c} represents the sequencing read for the alleged offspring,
D_{af} represents the sequencing read for the second tested subject,
gₘ represents the genotype for a corresponding locus of the first tested subject,
g_{c} represents the genotype for a corresponding locus of the alleged offspring,
g_{af} represents the genotype for a corresponding locus of the second tested subject,
T(g_{c}|gₘ, g_{af}) represents a likelihood that both alleles of the alleged offspring are inherited from the first and second tested subjects, and
T(g_{c}|g_{mf}) represents a likelihood that the tested second subject is not biologically related to the alleged offspring.

16. The system of claim 15, wherein the first tested subject is a mother of the offspring and the second tested subject is an alleged father of the offspring.

17. A non-transitory computer readable medium for storing computer instructions that, when executed by one or more processors, causes the one or more processors to perform a next generation sequencing (NGS) based method for genetic testing, comprising:
applying, for one or more genetic loci, respective NGS data related to genotype of a first tested subject, genotype of a second tested subject, and genotype of an alleged offspring of the first and second tested subjects to a statistical model for calculating a value representing a likelihood that the alleged offspring is a true offspring of the first and second subjects; and
determining, based on the respective values calculated for the one or more genetic loci, a likelihood that the alleged offspring is a true offspring of the first and second tested subjects;
wherein the NGS data includes:
genotype and sequencing read of the first tested subject;
genotype and sequencing read of the second tested subject; and
genotype and sequencing read of the alleged offspring;
wherein the statistical model utilizes:
a probability of the genotype of the first tested subject in a subject population;
a probability of the genotype of the second tested subject in a subject population; and
a probability of the genotype of the alleged offspring in a subject population.

18. The non-transitory computer readable medium of claim 17, wherein the statistical model utilizes the respective probability of the genotype of the first tested subject, the second tested subject, and the alleged offspring as posterior probability with the sequencing read of the first tested subject, the second tested subject, and the alleged offspring.

19. The non-transitory computer readable medium of claim 17, wherein the statistical model applies the following for calculating the value of the respective genetic loci:
$$\frac{{\sum }_{{g}_{c} ; {g}_{m} , {g}_{af}} T ( {g}_{c} \vert {g}_{m} , {g}_{af} ) P ( {g}_{c} \vert {D}_{c} ) P ( {g}_{m} \vert {D}_{m} ) P ( {g}_{af} \vert {D}_{af} )}{{\sum }_{{g}_{c} , {g}_{m}} T ( {g}_{c} \vert {g}_{m} ) P ( {g}_{c} \vert {D}_{c} ) P ( {g}_{m} \vert {D}_{m} )}$$
where
Dₘ represents the sequencing read for the first tested subject,
D_{c} represents the sequencing read for the alleged offspring,
D_{af} represents the sequencing read for the second tested subject,
gₘ represents the genotype for a corresponding locus of the first tested subject,
g_{c} represents the genotype for a corresponding locus of the alleged offspring,
g_{af} represents the genotype for a corresponding locus of the second tested subject,
T(g_{c}|gₘ, g_{af}) represents a likelihood that both alleles of the alleged offspring are inherited from the first and second tested subjects, and
T(g_{c}|g_{mf}) represents a likelihood that the tested second subject is not biologically related to the alleged offspring.

20. The non-transitory computer readable medium of claim 17, wherein the first tested subject is a mother of the offspring and the second tested subject is an alleged father of the offspring.
